(19) United States
(12) Patent Application Publication
(10) Pub. No.: US 2002/0164376 A1
(43) Pub. Date: Nov. 7, 2002

(54) Microparticles and their use in cancer treatment

(76) Inventors: Martin M. Eatock, Glasgow (GB); Roy Harris, Nottingham (GB)

Correspondence Address: Madeline I. Johnston, Morrison & Foerster LLP, 755 Page Mill Road, Palo Alto, CA, 94304-1018, US

(21) Appl. No.: 10/172,395

(22) Filed: Jun. 14, 2002

Related U.S. Application Data
(63) Continuation of application No. 09/015,964, filed on Jan. 30, 1998, now abandoned.

(30) Foreign Application Priority Data
Aug. 2, 1997 (GB) 9716289.5
Jan. 30, 1997 (GB) 9701876.6

(51) Int. Cl. A61K 39/395; A61K 33/24; A61K 31/704

(52) U.S. Cl. 424/499; 424/649; 424/178.1; 514/21; 514/34

(57) ABSTRACT

A method of treating tumors of a tissue selected from liver, lung, spleen and peritoneum, comprises administering to a subject hosting the tumors microparticles having a size appropriate to target the tissue, the microparticles having at least one cytotoxic agent bound thereto. Microparticles having two bound cytotoxic agents are new. The microparticles are also useful for treating multi drug resistance.

### FIELD OF THE INVENTION

This invention relates to microparticles and to their use in cancer therapy.

### BACKGROUND OF THE INVENTION

In cancer therapy using cytotoxic agents, it is desirable to localise the effect of the drug. It is also desirable to ensure that the drug remains at the site of action. Achieving these aims is difficult.

Another problem associated with cancer therapy is where the tumour exhibits multi-drug-resistance (MDR). This is often found following partially successful chemotherapy.

Microparticles, their production by spray-drying, and their utility as drug carriers, are disclosed in WO-A-9218164, WO-A-9609814 and WO-A-9618388. In particular, WO-A-9618388 describes microparticles, typically of albumin, additionally comprising a cytotoxic or other therapeutic agent. The microparticles are produced by spray-drying, under conditions allowing good size control, and are then stabilised, e.g. by heating, before the therapeutic agent is coupled via retained functional groups on the microparticles. Specifically, microparticles having a median size of about 3 μm, with bound methotrexate, FUDR or doxorubicin are shown to have utility in a rat liver tumour model, in vitro. This model shows retention of cytotoxic activity only, and gives no predictive indication as to suitable sites of action in vivo.

It is known to use carrier materials in order to target cytotoxic drugs to the site of action. Typically, microparticles or other such materials comprise a matrix in which the drug is entrapped.

### SUMMARY OF THE INVENTION

According to one aspect of this invention, it has now been found that microparticles having a bound cytotoxic agent, of the type described in WO-A-9618388, have remarkable and surprising utility in the treatment of certain tumours, specifically of the spleen, lung or, especially, liver. The present invention takes advantage of the fact that microparticles of a particular size can be adapted for relatively specific administration to a particular site of action. Thus, for example, the particles should have a median size of 1-5 μm for administration to the liver, above 6 μm for administration to the lung, and 1-5 μm for administration to the spleen, if appropriate with means to bypass the liver. It has been found that, over and above this effect, such microparticles will not only accumulate in a desired tissue, but persist at this locus, become localised around tumour tissue and without even distribution throughout healthy tissue, thereby providing unexpectedly focused tumour treatment. The data presented below show, inter alia, the highly targeted delivery to tumour tissue within the liver and persistence of the cytotoxic-loaded microparticles at that locus for at least 14 days, and the effect that is achieved during residence. These remarkable effects have been observed both in mice and rats.

According to another aspect of the present invention, microparticles carrying two cytotoxic drugs, or one such drug and a targeting and/or echogenic agent, are useful to overcome tumour resistance to such drugs, including MDR. Microparticles carrying two or more drugs are new.

### DESCRIPTION OF THE INVENTION

Without wishing to be bound by theory, it appears that cytotoxicity is related to the uptake of albumin-based materials by cells of certain tumour types. The microspheres may provide a useful delivery vehicle for intra-cavitary treatment, for example of ovarian carcinoma.

It has been suggested that the expression of the cell membrane efflux pump P-glycoprotein may be responsible for inducible resistance to drugs, including doxorubicin, in a number of human cancers. The novel drug delivery system may have the ability to increase targeting of therapy and may overcome P-glycoprotein-mediated resistance and/or down-regulation of topoisomerase II, perhaps by enhancing intracellular drug retention and overwhelming the mechanisms.

Again without wishing to be bound by theory, it is possible that the results that have been observed are the consequence of the microcapsules being taken up by Kupffer cells which act as a vehicle to the locus of action. If this theory is correct, the same effect may be observed in other tissues having analogous functionality to Kupffer cells, i.e. macrophages of the organisms in the endothelial system.

This invention therefore provides targeted and effective cancer therapy. This may be achieved by systemic or regional delivery, and can achieve tumour eradication, e.g. of liver primaries or secondaries.

Microparticles may be prepared by the procedures described in WO-A-9218164, WO-A-9609814 and WO-A-9618388. These spray-drying and associated particle manipulation processes enable the production of protein microcapsules with defined size distribution, e.g. of up to 10 μm in diameter. For example, the microparticles may be predominantly 0.1 to 10 μm in size, or of submicron size.

Both soluble and insoluble (cross-linked) biologically-active protein microcapsules can be produced, depending on the processing method. Suitable “wall-forming materials” are described in WO-A-9218164. A preferred material is HSA (human serum albumin).

The microparticles of this invention may have the physical characteristics described in the three publications identified above, e.g. being biodegradable, smooth and spherical. Known conditions can be used to produce, for example, microcapsules of 1-5 μm, e.g. c.4 μm diameter.

The cytotoxic agents, or drug and targeting agent, are then covalently bound to the microparticles. This is described in more detail in WO-A-9609814; as also described there, spray-dried microparticles may retain functional groups available for the binding of therapeutic agents.

Suitable targeting agents are known. The particles may themselves act to this end, e.g. if of an appropriate size.

Cytotoxic drugs that may be used in the invention will be readily apparent to one of ordinary skill in the art. Choice will depend on the condition to be treated. The cytotoxic agent may be, for example, doxorubicin, mitomycin, cisplatin, methotrexate or 5-fluoro-2′-deoxyuridine (FUDR). These may be loaded at levels of up to 20% w/w, e.g., respectively, 1%, 1%, 4-8%, 17% and 7%, w/w.

In certain circumstances, e.g. for the treatment of multi-drug resistance, it may be desirable to use two cytotoxic agents.

Covalent attachment of the drug to the microcapsule is in contrast to systems that trap drug in the matrix. There may be attachment of a variety of drugs using different cross-linkers (such as EDC) and native binding sites on HSA (OH, NH₂, COOH and, for cisplatin, the SH groups). Because of the different binding site available for another active material, e.g. for doxorubicin, cisplatin is a preferred choice for one such material. Different agents may also be chosen because of their different mechanisms of action, or different release rates.

The mechanism of drug loading allows the same microcapsules to be loaded with two (or more) drugs, perhaps using different mechanisms. An example would be doxorubicin and cisplatin loaded on the same microcapsules. Alternatively, microcapsules with different drugs as the pay load could simply be mixed, if cells take up more than one microcapsule. It is generally preferred to use one microcapsule, and therefore the use of loading with more than one drug is desirable if that type of therapy is required.

In either case, the drug-resistant cells may be presented simultaneously with more than one cytotoxic drug. Likewise, the individual tumour cell may be presented with cytotoxic drug simultaneously with another agent such as a cytokine, or a targeting agent such as an antibody. For example, the observed resistance to cisplatin by ovarian carcinomatosis may be overcome by the use of microparticles carrying cisplatin and doxorubicin, by virtue of the much higher cellular cisplatin level and the lethally high doxorubicin level.

The drug-loaded microparticles may be formulated for use in any conventional manner appropriate for administration such that the active agent can reach the locus of action. The amount of active agent to be administered in treating a patient will be chosen according to, inter alia, the nature of the agent, the condition of the subject and the severity of the tumour, as will be evident to one of ordinary skill in the art. For example, a known amount of a known drug may be given, or an amount calculated on the basis of the Examples. It is an advantage of the invention that the active agent accumulates and persists in the region of tumour tissue, and this should enable reduced dosages to be administered, thereby reducing side-effects for a given dose of the cytotoxic agent. Unit dose formulations may be provided, adapted to deliver all or part of this dosage range, e.g. 1 to 4 times daily. It is an advantage of this invention that many fewer doses can be used, e.g. weekly or even monthly, because of the persistence and localisation that may be observed.

Microparticles of this invention are primarily intended for intra-cavitary treatment. For this purpose, they may be administered directly, intraperitoneally or, using relatively small particles, intravenously. They may be formulated with any suitable carrier. Intraperitoneal administration is usually unsuitable for cytotoxic agents, but the localised effect of the present invention means that lower doses can be used.

As explained above, the preparation of microparticles having one bound cytotoxic agent is known. See, in particular, Examples 5-7 of WO-A-9618388. The preparation of microcapsules carrying two such agents may be achieved by analogy; a specific illustration is provided in Example 1. Subsequent Examples illustrate the utility of the invention. HSAMs=human serum albumin microcapsules.

### EXAMPLE 1

HSAMs (100 mg) were sunk for 30 minutes in 1% Tween 80 solution and were then washed with distilled water (3×5 ml) to remove Tween and excipient. The microcapsules were resuspended in 2.1 ml cisplatin solution (1 mg/ml, Faulding Pharmaceuticals) and the reaction was stirred for four days at room temperature in the absence of light.

The microcapsules were washed in distilled water (4×5 ml) to remove any unbound cisplatin, and collected by centrifugation. Doxorubicin (3 mg) and EDC (6 mg) were added in a total volume of 1 ml distilled water and the mixture was stirred at 37° C. for 20 hours. The microcapsules were centrifuged and washed in distilled water until the supernatant was clear of unreacted doxorubicin.

The product was resuspended in 1 ml distilled water. A 5 mg sample was removed and digested with pepsin (10% w/w) in 1M HCl. A comparison of the digest with a standard curve of doxorubicin using UV/VIS spectrophotometry at 495 nm revealed 0.96 moles of doxorubicin had been bound per mole of HSA. The cisplatin loading was determined using atomic absorption spectrometry, and was found to be 2-3%.

### EXAMPLE 2

This experiment compares doxorubicin free drug and doxorubicin microcapsules in the MCF7 cell line and the related doxorubicin-resistant cell line MCF7/dox. It was noted that the doxorubicin-resistant cell line had a lower IC₅₀ with microcapsules compared with free drug, i.e. the microcapsule presentation reversed the drug resistance.

More specifically, the experiment compared the cytotoxicity of a novel preparation of doxorubicin covalently-linked to a human serum albumin microsphere carrier between 2 and 3 μm in diameter on a doxorubicin-sensitive human breast cancer cell line and its doxorubicin-resistant P-glycoprotein expressing daughter cell line. HSAMs were produced and heat-stabilised prior to incubation with 1-(3-dimethylaminopropyl)-3-ethylcarbodmiimide (EDC) and doxorubicin (Dox). The EDC “activates” exposed carboxyl residues on the HSAMs, allowing covalent binding of Dox amino sugar. The human MCF7 cell line and its doxorubicin-resistant daughter cell line, MCF7/Dox were used.

Cells were plated in 24 well plates at a concentration of 50,000 cells/well and incubated with either doxorubicin or a solution of doxorubicin-HSAMs at varying concentrations for 24 hours. The medium was then changed, cells were incubated for a further 72 hours before harvesting and counting with a Coulter Counter. The IC₅₀ for the MCF7 parent cell line with doxorubicin was 0.031 μg/ml (Standard error (SE)=0.002) whereas for the doxorubicin-resistant line it was 0.387 μg/ml (SE=0.049, p=0.002). 24 hour incubation of the doxorubicin-resistant cell line with the drug-loaded microspheres showed an IC₅₀ of 0.062 μg/ml (SE=0.037) (expressed as μg doxorubicin per ml), which was significantly lower than the IC₅₀ for doxorubicin in this cell line (p=0.006) and not significantly different from that seen in the parent cell line (p=0.45).

### EXAMPLE 3

FUDR-loaded HSAMs were administered by intraperitoneal injection to groups of tumour-bearing mice (C170HM₂). Thus, using a human colorectal tumour, the invasive effect on the cross-sectional area of liver tumours was observed. Dosing was after 32 days, and kill after 39 days. In Group 1 (untreated controls), 7 tumours, up to 2000 mm² in area, were observed. In the other groups, respectively dosed with 0.64, 1.28 and 2.00 mg/kg (each n=2) of the loaded HASMs, there were no or reduced tumours; the reduced tumours had areas of no more than 500 mm² (or slightly more in the last group).

### EXAMPLE 4

Doxorubicin-loaded HSAMs were administered by introperitoneal injection to groups of tumour-bearing mice (C170HM₂), at 0.08, 0.16 or 0.24 mg/kg (each n=2), at day 32. Following termination at day 39, the effect of administration on the liver was observed. Except in 1 or 2 cases, where tumours weights were 3-4 g, the remainder of the tumours had disappeared. In an untreated control group, several tumours were found, weighing 0.1 to 1.5 g.

With fluorescent labelling, signal was detectable at 7 days post-dosing. This clearly indicates surprising accumulation, persistence and localised effect, in addition to efficacy and lack of acute toxicity.

### EXAMPLE 5

0.24 mg/kg doxorubicin-loaded HSAMs (drug loading approx. 1% w/w) were administered to a group of tumour-bearing mice (C170HM₂). A further group received HSAMs, at a protein concentration of 100 mg/ml, as a control. A third group received 0.25 mg/kg free doxorubicin. There were 12 mice per group. Dosing was at day 27, termination at day 41.

For the control, the mean liver tumour weight was c. 1.3 g. Following administration of free doxorubicin, the mean weight was c. 0.3 g. Using the method of the invention, no tumours were observed.

1. A method of treating tumours of a tissue selected from liver, lung, spleen and peritoneum, which comprises administering to a subject hosting the tumours microparticles having a size appropriate to target the tissue, the microparticles having at least one cytotoxic agent bound thereto.

2. A method according to claim 1, wherein the tissue is liver and the median size of the microparticles is 1-5 μm.

3. A method according to claim 1 or claim 2, wherein the cytoxic agent is selected from methotrexate, doxorubicin, cisplatin and FUDR.

4. A method according to any preceding claim, wherein the microparticles are of human serum albumin.

5. A method according to any preceding claim, wherein the tissue is liver, lung or spleen.

6. A method according to claim 5, wherein the tissue is liver.

7. Microparticles comprising two or more cytotoxic agents bound thereto.

8. Microparticles according to claim 7, which are 0.1 to 10 μm in diameter.

9. Microparticles according to claim 7 or claim 8, of human serum albumin.

10. Microparticles according to any of claims 7 to 9, wherein the cytotoxic agents are doxorubicin and cisplatin.

11. A method of treating multi-drug-resistant tumours, which comprises administering to the subject microparticles according to any of claims 7 to 10.

12. A method of treating multi-drug-resistant tumours, which comprises administering to the subject microparticles having a cytotoxic agent and a targeting agent.

13. A method according to any of claims 1 to 6, wherein the microparticles are as defined in any of claims 7 to 10.
